(19) United States
(12) Patent Application Publication
(10) Pub. No.: US 2017/0143405 A1
(43) Pub. Date: May 25, 2017

(54) APPARATUSES, SYSTEMS AND METHODS FOR TREATING ULCERATIVE COLITIS AND OTHER INFLAMMATORY BOWEL DISEASES

(71) Applicant: COVIDIEN LP, Mansfield, MA (US)

(72) Inventors: KATHY E. ROOKS, LONGMONT, CO (US); ROBERT M. SHARP, BOULDER, CO (US); JAMES D. ALLEN, IV, BROOMFIELD, CO (US)

(21) Appl. No.: 15/243,483

(22) Filed: Aug. 22, 2016

Related U.S. Application Data
(60) Provisional application No. 62/258,025, filed on Nov. 20, 2015.

(51) Int. Cl. A61B 18/14

(52) U.S. Cl. CPC A61B 18/14; A61B 2018/1435; A61B 2018/1467; A61B 2018/00494

(57) ABSTRACT

An apparatus for treating inflammatory bowel diseases is provided. The apparatus includes a body and an elongate spring coil extending distally from the body and configured to be positioned within a patient's colon. The elongate spring coil defines a spiral configuration and is configured to be selectively moved relative to the body. The apparatus also includes at least one electrode disposed on the elongate spring coil and configured to emit energy. The at least one electrode is moved along a spiral path in response to movement of the elongate spring coil relative to the body. A method of use for treating inflammatory bowel diseases is also provided.

### CROSS REFERENCE TO RELATED APPLICATION

The present application claims the benefit of and priority to U.S. Provisional Application Ser. No. 62/258,025, filed on Nov. 20, 2015, the entire contents of which are incorporated herein by reference.

### BACKGROUND

Technical Field

The present disclosure relates to treatment of Inflammatory Bowel Diseases (IBDs) and, more particularly, to surgical apparatuses, systems, and methods for treating ulcerative colitis and other IBDs, e.g., Crohn's Disease.

Background of Related Art

Ulcerative colitis, an IBD, is a disease of the colon in which inflammation and ulcers, or sores, form on the interior wall of the colon. Ulcerative colitis manifests itself, usually intermittently and at varying degrees of severity, in symptoms such as stomach pain, diarrhea, and/or bloody stool. Typically, anti-inflammatory medication(s) are prescribed for patients suffering from ulcerative colitis. In extreme cases, chronic cases, or cases in which medication(s) fails to adequately treat the patient's symptoms, surgery to remove all or part of the diseased portions of the rectum and/or colon may be performed.

Although medication(s) and surgical removal of diseased portions of the rectum and/or colon are effective in certain instances, there is a need for surgical apparatuses, systems, and methods to more effectively and/or efficiently treat ulcerative colitis and other IBDs, e.g., Crohn's Disease, while minimizing side effects and damage to un-diseased tissue.

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

An apparatus for treating inflammatory bowel diseases provided in accordance with the present disclosure includes a body and an elongate spring coil extending distally from the body and configured to be positioned within a patient's colon. The elongate spring coil defines a spiral configuration and is configured to be selectively moved relative to the body. The apparatus also include at least one electrode disposed on the elongate spring coil and configured to emit energy. The at least one electrode is moved along a spiral path in response to movement of the elongate spring coil relative to the body.

In aspects, the body includes a crank. The crank is operatively coupled to the elongate spring coil and configured to move the elongate spring coil relative to the body upon manual turning of the crank relative to the body.

In some aspects, the body includes a first switch disposed thereon and operably coupled to the at least one electrode. Activating the switch energizes the at least one electrode.

In some aspects, the at least one electrode is a monopolar electrode.

According to another aspect of the present disclosure, an apparatus for treating inflammatory bowel diseases includes a body and a flexible elongate member extending distally from the body. The flexible elongate member is configured to be positioned within a patient's colon. The apparatus also includes a plurality of electrodes coupled to the flexible elongate member and biased radially outwardly relative to the flexible elongate member such that the plurality of electrodes are maintained in contact with an inner surface of a patient's colon upon movement of the flexible elongate member therethrough.

In some aspects, the plurality of electrodes are monopolar electrodes or bipolar electrodes.

In certain aspects, the plurality of electrodes includes a first electrode and a second electrode spaced-apart along the flexible elongate member. The first and second electrodes each define a disc-shaped configuration and are configured for positioning against an inner surface of a patient's colon in circumferential contact therewith.

In some aspects, the first electrode is configured to be charged to a first potential and the second electrode is configured to be charged to a second potential for conducting bipolar energy therebetween.

In some aspects, a diameter of the first electrode is different from a diameter of the second electrode.

In other aspects, the plurality of electrodes includes first and second bundles of electrically-conductive spring members spaced-apart along the elongate flexible member. The first and second bundles of spring members are configured for positioning against an inner surface of a patient's colon in circumferential contact therewith.

In certain aspects, the first bundle of electrically-conductive spring members is configured to be charged to a first potential and the second bundle of electrically-conductive spring members is configured to be charged to a second potential for conducting bipolar energy therebetween.

In other aspects, the flexible elongate member defines a hollow interior and at least one aperture therethrough. The plurality of electrodes are selectively deployable relative to the flexible elongate member from a retracted position. The plurality of electrodes are disposed within the hollow interior of the flexible elongate member, and an extended position. The plurality of electrodes extend through the at least one aperture and from the flexible elongate member in the extended position.

In other aspects, the plurality of electrodes are arranged in a plurality of groups of electrodes and the groups of electrodes spaced-apart along the elongate flexible member. Adjacent groups of electrodes are configured to define different polarities for conducting bipolar energy therebetween.

In certain aspects, the apparatus further includes a cooling system having a fluid line coupled to a source of fluid and extending through the flexible elongate member to a distal end thereof. The flexible elongate member includes a nozzle disposed at the distal end thereof and coupled to a distal end of the fluid line. The nozzle configured to expel fluid therefrom.

In aspects, the body includes a first switch disposed thereon and operably coupled to the plurality of electrodes. Activating the first switch energizes the plurality of electrodes.

In aspects, the body includes a second switch disposed thereon operably coupled to the nozzle. Activating the second switch causes the nozzle to release fluid therefrom.

A method of use for treating inflammatory bowel diseases is provided in accordance with the present disclosure includes advancing a flexible elongate member having at least one electrode coupled thereto into a patient's colon. The method of use further includes positioning the flexible elongate member such that the at least one electrode is disposed in contact with tissue of an inner surface of a patient's colon. The method of use further includes energizing the at least one electrode, and moving the flexible elongate member through a patient's colon while maintaining the at least one electrode in contact with tissue to treat tissue with the at least one electrode.

In aspects, the method of use further includes a cooling system configured to cool tissue treated by the at least one electrode.

In certain aspects, the method of use further includes at least one electrode movably attached to the flexible elongate member, and placing the at least one electrode includes deploying the at least one electrode from a retracted position to an extended position.

In aspects, the method of use further includes at least one electrode moved linearly or along a spiral path.

### BRIEF DESCRIPTION OF THE DRAWINGS

Various aspects and features of the present disclosure described herein with reference to the drawings wherein:

FIG. 1 is a schematic illustration of a gastrointestinal system of a patient, showing the stomach, small intestine, large intestine, colon, and rectum;

FIG. 2A is a side view of a treatment apparatus provided in accordance with the present disclosure;

FIG. 2B is a schematic illustration of the treatment apparatus of FIG. 2A positioned within the colon of the gastrointestinal system of FIG. 1, in a first position;

FIG. 2C is a schematic illustration of the treatment apparatus of FIG. 2A positioned within the colon of the gastrointestinal system of FIG. 1, in a second position;

FIG. 3A is a side view of another treatment apparatus provided in accordance with the present disclosure;

FIG. 3B is a schematic illustration of the treatment apparatus of FIG. 3A positioned within the colon of the gastrointestinal system of FIG. 1;

FIG. 4A is a side view of another treatment apparatus provided in accordance with the present disclosure;

FIG. 4B is a schematic illustration of the treatment apparatus of FIG. 4A positioned within the colon of the gastrointestinal system of FIG. 1;

FIG. 5A is a side view of another treatment apparatus provided in accordance with the present disclosure;

FIG. 5B is a schematic illustration of the treatment apparatus of FIG. 5A positioned within the colon of the gastrointestinal system of FIG. 1;

FIG. 6A is a side view of another treatment apparatus provided in accordance with the present disclosure, in a retracted state;

FIG. 6B is an enlarged, side, cross-sectional view of the area of detail indicated as “6B” in FIG. 6A;

FIG. 6C is a side view of the treatment apparatus of FIG. 6A, in an expanded state;

FIG. 6D is an enlarged, side, cross-sectional view of the area of detail indicated as “6D” in FIG. 6C;

FIG. 6E is a schematic illustration of the treatment apparatus of FIG. 6A positioned within the colon of the gastrointestinal system of FIG. 1 in the retracted state and; and

FIG. 6F is a schematic illustration of the treatment apparatus of FIG. 6A positioned within the colon of the gastrointestinal system of FIG. 1 in the expanded state.

### DETAILED DESCRIPTION

As IBD's such as ulcerative colitis may only affect portions of the colon, it is desirable to focus treatment towards such diseased areas while limiting damage to surrounding tissue and critical structures. Accordingly, the present disclosure provides various apparatuses, systems, and methods configured to facilitate the focused or controlled energy-based treatment of diseased portions of the colon while limiting damage to surrounding portions of the colon and other surrounding tissue and critical structures. The various embodiments of the present disclosure may be implemented using a variety of types of energy, e.g., RF, microwave, ultrasonic, optical, thermal, etc. Further, although the various apparatuses, systems, and methods provided herein may be utilized to treat any suitable type of diseased tissue, particular reference will be made to the colon “C.”

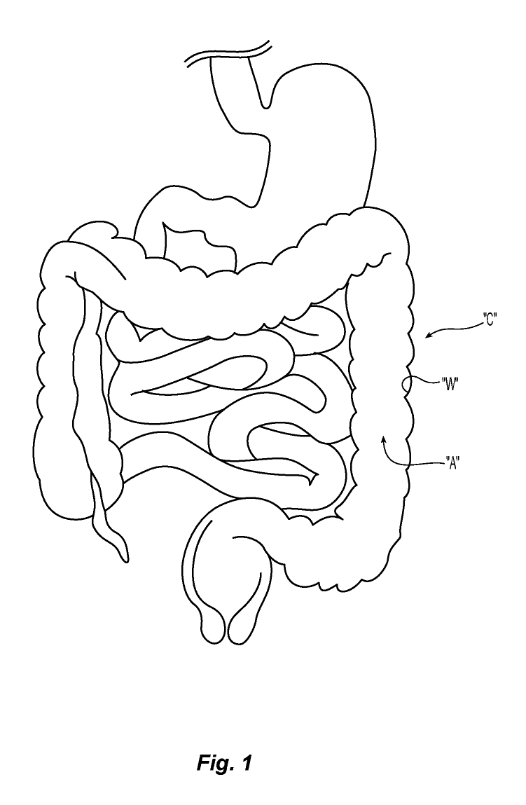

With reference to FIG. 1, a schematic illustration of a gastrointestinal system of a patient, generally, showing the stomach, small intestine, large intestine, colon, and rectum. The colon “C,” more specifically, has an inner wall “W” that defines an annular area “A.”

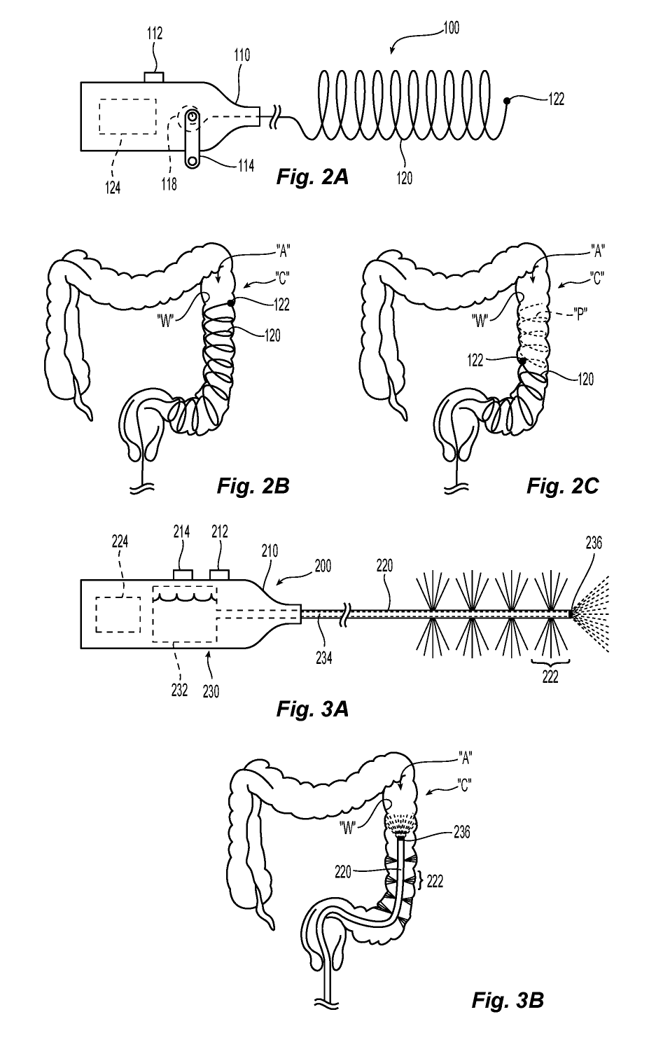

With reference to FIGS. 2A-2C, a treatment apparatus provided in accordance with the present disclosure and configured to treat diseased bowel tissue in the colon “C” is shown generally identified as reference numeral 100.

Treatment apparatus 100 generally includes a body 110 having a switch 112 and a manual crank 114 disposed thereon. Enclosed in body 110 is a spool 118 operably coupled to manual crank 114 to enable manual rotation thereof, although powered rotation of spool 118, e.g., via a motor, is also contemplated. An elongate spring coil 120 is coupled to spool 118 and is configured to either wind around or unwind from spool 118 in response to rotation of spool 118. Spring coil 120 may be formed of any flexible and electrical conductive material including surgical steel. The length of spring coil 120 is covered by an insulative layer (not shown), which may be formed of a polytetrafluoroethylene (PTFE). However, any other suitable non-conductive material may be used. Spring coil 120 extends distally from body 110 and forms a helix or spiral configuration.

An electrode 122 is attached to the distal end of spring coil 120. Further, additional electrodes (not shown) may be disposed along the length of spring coil 120. Electrode 122 defines a spherical configuration, although other configurations are also contemplated. Electrode 122 is configured to be energized and to emit energy therefrom, e.g., monopolar energy to be returned via a remote return pad (not shown).

Electrode 122 is electrically coupled to a generator 124 disposed within body 110 via spring coil 120. Switch 112 disposed on body 110 is disposed between and in electrical communication with generator 124 and electrode 122 for enabling the selective supply of energy to electrode 122. Switch 112 may be an on/off switch or an adjustable knob that allows the operator to adjust an amount, duration, and/or frequency of energy supplied to electrode 122. Generator 124 may alternatively be configured to provide microwave energy or any other suitable form of energy to electrode 122. Although generator 124 has been described above as being incorporated into body 110 of treatment apparatus 100, an external generator may alternatively be provided.

In a method of use, as shown in FIG. 2B, treatment apparatus 100 is initially positioned such that spring coil 120 and electrode 122 are positioned within an annular area “A” of a patient's colon “C” to be treated such that electrode 122 is disposed at the furthest upstream position relative to the diseased area of tissue to be treated. Spring coil 120 is configured to define a sufficient diameter such that electrode 122 is biased into contact with the inner wall “W” of the colon “C.” Once this position has been achieved, switch 112 may be activated to supply energy to electrode 122 for treating tissue in contact therewith and adjacent thereto.

As shown in FIG. 2C, upon turning of crank 114, spring coil 120 is wound around spool 118 thereby pulling spring coil 120 downstream through the colon “C” towards body 110 of treatment apparatus 100. Due to the spiral configuration of spring coil 120, pulling spring coil 120 through the colon “C” in this manner causes spring coil 120 and electrode 122 to move through the colon “C” along a spiral path “P,” thus treating a full circumference of the annular area “A” of tissue to be treated. As a result of the bias of spring coil 120 detailed above, electrode 122 remains in contact with the inner wall “W” of the patient's colon “C” during the movement thereof.

Now turning to FIGS. 3A and 3B, another treatment apparatus provided in accordance with the present disclosure and configured to treat diseased tissue in the colon “C” is shown generally identified as reference numeral 200.

Treatment apparatus 200 generally includes a body 210 having a first switch 212 and a second switch 214 disposed thereon. An elongate hollow tube 220 is coupled to body 210 and extends distally therefrom. Elongate hollow tube 220 may be formed of any suitable flexible and non-conductive material, for example, polymeric materials. Attached to hollow tube 220 and extending radially therefrom are electrodes 222. Electrodes 222 may be formed as a plurality of groups of electrode probes spaced-apart along hollow tube 220 and defining any suitable cross-sectional shape. Each electrode 222 extends radially outwardly from hollow tube 220. Further, the electrodes 222 forming each group may extend in different directions so as to extend from hollow tube 220 circumferentially about hollow tube 220. Electrodes 222 may be configured to conduct monopolar energy to a remote return pad (not shown) or may be configured such that the electrodes 222 of adjacent groups of electrode probes are energized to different potentials for conducting bipolar energy therebetween. Electrodes 222 may be formed from any suitable material and may be resiliently flexible to facilitate contacting and maintaining contact with the colon “C,” including metal, e.g., copper, gold, silver, etc.

Electrodes 222 are electrically coupled to a generator 224 disposed within body 210. A first switch 212 disposed on body 210 is disposed between and in electrical communication with generator 224 and electrodes 222 for enabling the selective supply of energy to electrodes 222. Switch 212 may be an on/off switch or an adjustable knob that allows the operator to adjust an amount, duration, and/or frequency of energy supplied to electrodes 222. Generator 224 may alternatively be configured to provide microwave energy or any other suitable form of energy to electrodes 222. Although generator 224 has been described above as being incorporated into body 210 of treatment apparatus 200, an external generator may alternatively be provided.

Treatment apparatus 200 further includes a cooling system 230. Cooling system 230 includes a fluid reservoir 232 that may be positioned external to body 210, positioned on body 210, or positioned within body 210. In embodiments where fluid reservoir 232 is external to body 210, body 210 includes an inflow port (not shown) to provide fluid communication between fluid reservoir 232 and a fluid line 234. Fluid reservoir 232 is configured to hold a supply of cooling fluid capable of conductively and/or conventionally absorbing heat from the treated tissue. Exemplary cooling fluids include but are not limited to water and saline.

Fluid line 234 is coupled to fluid reservoir 232 and extends distally through hollow tube 220, terminating at a nozzle 236 positioned on the distal end of hollow tube 220. Alternatively or additionally, nozzles disposed along the length of hollow tube 220 are also contemplated. Cooling system 230 may also include a pump (not shown) to pump cooling fluid from fluid reservoir 232 through fluid line 234 such that cooling fluid is expelled through nozzle 236 under pressure. Second switch 214 is operatively connected to cooling system 230 such that cooling system 230 may be selectively operated by a clinician.

In a method of use, as shown in FIG. 3B, hollow shaft 220 is positioned within the annular area “A” of the patient's colon “C” such that electrodes 222 are disposed in contact with the diseased area of tissue to be treated. Hollow shaft 220 and electrodes 222 are dimensioned such that electrodes 222 are biased into and maintained in contact with the inner wall “W” of the colon “C.” Once positioned in this manner, first switch 212 may be activated to energize electrodes 222 to treat tissue in contact therewith and adjacent thereto. Apparatus 200 may then be manipulated to draw hollow shaft 220 through the patient's colon “C,” thereby treating tissue along the inner wall “W” of the patient's colon “C.” Electrodes 222 remain in contact with inner wall “W” of the patient's colon “C” during the procedure and, as mentioned above, may be arranged circumferentially about hollow shaft 220 to treat a full circumference of the annular area “A” of tissue to be treated. Continuously or intermittently during use, second switch 214 may be activated to activate cooling system 230 to expel the cooling fluid to cool the treated tissue.

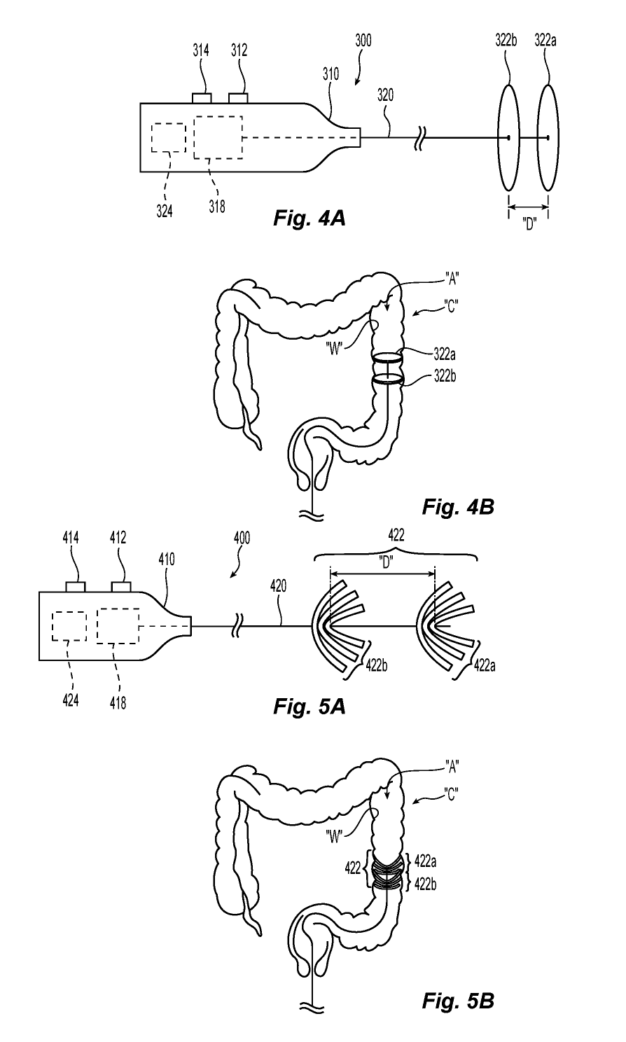

Referring to FIGS. 4A and 4B, another treatment apparatus provided in accordance with the present disclosure and configured to treat diseased tissue in the colon “C” is shown generally identified as reference numeral 300. Treatment apparatus 300 generally includes a body 310 having a first switch 312 and a second switch 314 disposed thereon. Enclosed in body 310 is a motor 318 having a spool (not shown) associated therewith.

An electrically conductive cable 320 is coupled to the spool (not shown) of the motor 318 and is configured to either wind around or unwind from the spool (not shown) upon activation of motor 318. Alternatively, conductive cable 320 may be fixed relative to body 310. Cable 320 extends distally from body 310 and may be formed of any flexible and conductive material. The length of cable 320 is covered by an insulative layer (not shown), which may be formed of a polytetrafluoroethylene (PTFE). However, any other suitable non-conductive material may be used.

A first electrode 322a is attached to a distal end of cable 320 and a second electrode 322b is disposed about cable 320 and positioned proximal to first electrode 322a, defining a distance “D” therebetween. First and second electrodes 322a, 322b may form a solid disc, however, a ring, hoop, or annulus configuration may also be provided. The peripheries of electrodes 322a, 322b may form a dull or rounded edge to prevent puncturing of tissue; however, electrodes 322a, 322b may also be used to scrape the inner wall “W” of the colon “C.”

First and second electrodes 322a, 322b are electrically coupled to cable 320 which, in turn, is electrically coupled with a generator 324. First switch 312 disposed on body 310 is disposed between and in electrical communication with generator 324 and first and second electrodes 322a, 322b for enabling the selective supply of energy to first and second electrodes 322a, 322b. Switch 312 may be an on/off switch or an adjustable knob that allows the operator to adjust an amount, duration, and/or frequency of energy supplied to first and second electrodes 322a, 322b. Generator 324 may alternatively be configured to provide microwave energy or any other suitable form of energy to first and second electrodes 322a, 322b. Although generator 324 has been described above as being incorporated into body 310 of treatment apparatus 300, an external generator may alternatively be provided.

First and second electrodes 322a, 322b may be configured to conduct monopolar energy to a remote return pad (not shown) or may be configured such that the first and second electrodes 322a, 322b are energized to different potentials for conducting bipolar energy therebetween. First and second electrodes 322a, 322b may be formed from any suitable material and may be resiliently flexible to facilitate contacting and maintaining contact with the colon “C.”

In a method of use, as shown in FIG. 4B, cable 320 and electrodes 322a, 322b are positioned within the annular area “A” of the patient's colon “C” such that electrodes 222 are disposed in contact with the diseased area of tissue to be treated. Cable 320 and electrodes 322a, 322b are dimensioned such that electrodes 322a, 322b are biased into and maintained in contact with the inner wall “W” of the colon “C.” Once positioned in this manner, first switch 312 may be activated to energize electrodes 322 to treat tissue in contact therewith and adjacent thereto.

Upon activating second switch 314, cable 320 is wound around spool 318 thereby pulling electrodes 322a, 322b downstream through the colon “C” towards body 310 of treatment apparatus 300. Due to the disc configuration of electrodes 322a, 322b, pulling electrodes 322a, 322b through the colon “C” in this manner causes electrodes 322a, 322b to treat a full circumference of the annular area “A” of tissue to be treated. Electrodes 322a, 322b remain in contact with inner wall “W” of the patient's colon “C” during the procedure.

Referring to FIGS. 5A and 5B, another treatment apparatus 400 is provided in accordance with the present disclosure including a body 410 incorporating a motor 418 and having first and second switches 412, 414 disposed thereon. Treatment apparatus 400 is similar to treatment apparatus 300 (FIGS. 4A and 4B) and, thus, only the differences therebetween are discussed in detail below.

Treatment apparatus 400 includes a plurality of electrically-conductive spring members 422 that function as electrodes. The plurality of spring members 422 are separated into a first bundle 422a of spring members 422 attached to the distal end of cable 420 and a second bundle 422b of spring members 422 disposed about cable 420 and positioned proximal to first bundle 422a by a distance “D.” Each of the spring members 422 may be formed from any flexible conductive material, e.g., a thin strip of material.

Similar to treatment apparatus 300, cable 420 and first and second bundles 422a, 422b are electrically coupled to generator 424. First and second bundles 422a, 422b may be configured to conduct monopolar energy to a remote return pad (not shown) or may be configured such that the first and second bundles 422a, 422b are energized to different potentials for conducting bipolar energy therebetween. Further, as shown in FIG. 5B, the operation of treatment apparatus 400 is similar to treatment apparatus 300.

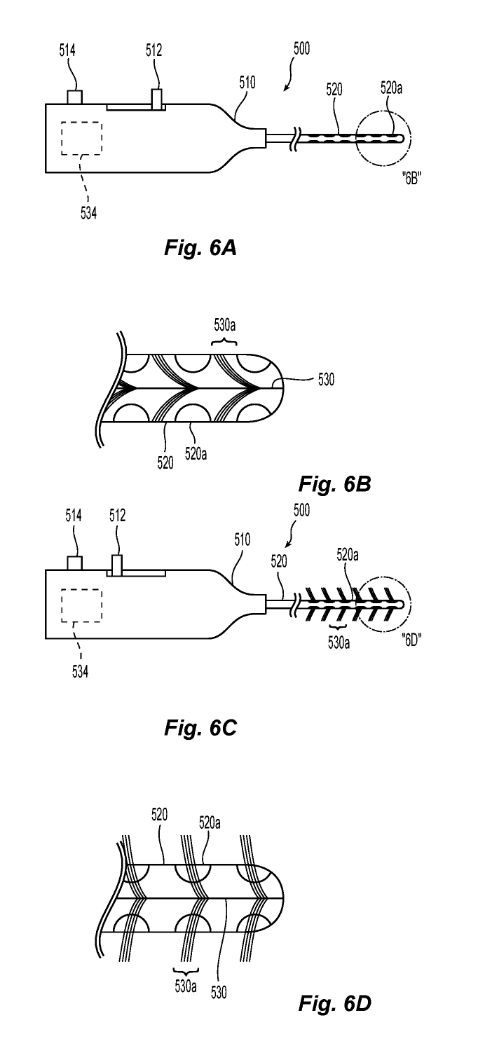

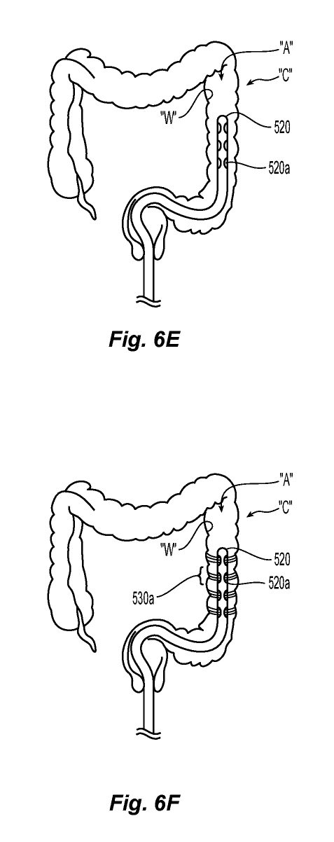

Now turning to FIGS. 6A-6F, another treatment apparatus provided in accordance with the present disclosure and configured to treat diseased tissue in the colon “C” is shown generally identified as reference numeral 500.

Treatment apparatus 500 includes a body 510 and an elongate hollow tube 520 that is coupled to and extends distally from body 510. Elongate hollow tube 520 may be formed of any suitable non-conductive material, for example, polymeric materials. Hollow tube 520 further defines a plurality of apertures 520a therethrough. Apertures 520a are disposed along the length of hollow tube 520 and radially thereabout.

Disposed within hollow tube 520, and extending therethrough, is a conductive cable 530 having clusters of electrodes 530a formed as probes extending outwardly from cable 530. Each cluster of electrodes 530a may include electrodes 530a extending in different directions so as to extend from the full circumference of cable 530.

A first switch 512 of body 510 is operatively connected to cable 530 and electrodes 530a. Cable 530 and electrodes 530a are initially positioned within hollow tube 520 such that electrodes 530a are aligned with apertures 520a of hollow tube 520. When first switch 512 is activated, e.g., slid longitudinally, cable 530 is pulled through hollow tube 520 such that electrodes 530a are moved from the retracted position (FIGS. 6A and 6B) to an extended position (FIGS. 6C and 6D). When electrodes 530a are in the extended position, they extend radially from cable 530 and hollow tubing 520, through apertures 520a thereof.

A second switch 514 disposed on body 510 is disposed between and in electrical communication with generator 524 and electrodes 530a for enabling the selective supply of energy to electrodes 530a. Second switch 514 may be an on/off switch or an adjustable knob that allows the operator to adjust an amount, duration, and/or frequency of energy supplied to electrodes 530a. Generator 524 may alternatively be configured to provide microwave energy or any other suitable form of energy to electrodes 530a. Although generator 524 has been described above as being incorporated into body 510 of treatment apparatus 500, an external generator may alternatively be provided.

In operation, as shown in FIGS. 6E and 6F, hollow tube 520 is positioned within the annular area “A” of the patient's colon “C.” First switch 512 is activated thereby moving electrodes 530a from the retracted position to the extended position. Hollow tube 520 and electrodes 530a are dimensioned such that electrodes 530a are biased into and maintained in contact with the inner wall “W” of the colon “C” when in the extended position. Once positioned in this manner, second switch 514 may be activated to energize electrodes 530a to treat tissue in contact therewith and adjacent thereto. Apparatus 500 may then be manipulated to draw hollow tube 520 through the patient's colon “C,” thereby treating tissue along the inner wall “W” of the patient's colon “C.” Electrodes 530a remain in contact with inner wall “W” of the patient's colon “C” during the procedure and, as mentioned above, may be arranged circumferentially about hollow tube 220 to treat a full circumference of the annular area “A” of tissue to be treated.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An apparatus for treating inflammatory bowel diseases, comprising:
a body;
an elongate spring coil extending distally from the body and configured to be positioned within a patient's colon, the elongate spring coil defining a spiral configuration and configured to be selectively moved relative to the body; and
at least one electrode disposed on the elongate spring coil and configured to emit energy, wherein the at least one electrode is moved along a spiral path in response to movement of the elongate spring coil relative to the body.

2. The apparatus according to claim 1, wherein the body includes a crank operatively coupled to the elongate spring coil and configured to move the elongate spring coil relative to the body upon manual turning of the crank relative to the body.

3. The apparatus according to claim 1, wherein the body includes a switch disposed thereon and operably coupled to the at least one electrode, wherein activating the switch energizes the at least one electrode.

4. The apparatus according to claim 1, wherein the at least one electrode is a monopolar electrode.

5. An apparatus for treating inflammatory bowel diseases, comprising:
a body;
a flexible elongate member extending distally from the body, the flexible elongate member configured to be positioned within a patient's colon; and
a plurality of electrodes coupled to the flexible elongate member and biased radially outwardly relative to the flexible elongate member such that the plurality of electrodes are maintained in contact with an inner surface of a patient's colon upon movement of the flexible elongate member therethrough.

6. The apparatus according to claim 5, wherein the plurality of electrodes are monopolar electrodes or bipolar electrodes.

7. The apparatus according to claim 5, wherein the plurality of electrodes includes a first electrode and a second electrode spaced-apart along the flexible elongate member, the first and second electrodes defining disc-shaped configurations and configured for positioning against an inner surface of a patient's colon in circumferential contact therewith.

8. The apparatus according to claim 7, wherein the first electrode is configured to be charged to a first potential and the second electrode is configured to be charged to a second potential for conducting bipolar energy therebetween.

9. The apparatus according to claim 7, wherein a diameter of the first electrode is different from a diameter of the second electrode.

10. The apparatus according to claim 5, wherein the plurality of electrodes includes first and second bundles of electrically-conductive spring members spaced-apart along the elongate flexible member, the first and second bundles of spring members configured for positioning against an inner surface of a patient's colon in circumferential contact therewith.

11. The apparatus according to claim 10, wherein the first bundle of electrically-conductive spring members is configured to be charged to a first potential and the second bundle of electrically-conductive spring members is configured to be charged to a second potential for conducting bipolar energy therebetween.

12. The apparatus according to claim 5, wherein the flexible elongate member defines a hollow interior and at least one aperture therethrough, and wherein the plurality of electrodes are selectively deployable relative to the flexible elongate member from a retracted position, wherein the plurality of electrodes are disposed within the hollow interior of the flexible elongate member, and an extended position, wherein the plurality of electrodes extend through the at least one aperture and from the flexible elongate member.

13. The apparatus according to claim 5, wherein the plurality of electrodes are arranged in a plurality of groups of electrodes, the groups of electrodes spaced-apart along the elongate flexible member, wherein adjacent groups of electrodes are configured to define different polarities for conducting bipolar energy therebetween.

14. The apparatus according to claim 13, further including a cooling system having fluid line coupled to a source of fluid and extending through the flexible elongate member to a distal end thereof, wherein the flexible elongate member includes a nozzle disposed at the distal end thereof and coupled to a distal end of the fluid line, the nozzle configured to expel fluid therefrom.

15. The apparatus according to claim 14, wherein the body includes a first switch disposed thereon and operably coupled to the plurality of electrodes, wherein activating the first switch energizes the plurality of electrodes.

16. The apparatus according to claim 15, wherein the body includes a second switch disposed thereon and operably coupled to the nozzle, wherein activating the second switch opens the nozzle to expel fluid therefrom.

17. A method of treating inflammatory bowel diseases, comprising:
advancing a flexible elongate member having at least one electrode coupled thereto into a patient's colon;
positioning the flexible elongate member such that the at least one electrode is disposed in contact with tissue of an inner surface of a patient's colon;
energizing the at least one electrode; and
moving the flexible elongate member through a patient's colon while maintaining the at least one electrode in contact with tissue to treat tissue with the at least one electrode.

18. The method according to claim 17, wherein the apparatus includes a cooling system configured to cool tissue treated by the at least one electrode.

19. The method according to claim 17, wherein the at least one electrode is movably attached to the flexible elongate member, and wherein placing the at least one electrode includes deploying the at least one electrode from a retracted position to an extended position.

20. The method according to claim 17, wherein the at least one electrode is moved linearly or along a spiral path.
